(19) United States
(12) Patent Application Publication
(10) Pub. No.: US 2024/0407406 A1
(43) Pub. Date: Dec. 12, 2024

(54) INCREASING HYDROLYSIS EFFICIENCY IN CEREAL PROCESS BY EMPLOYING LYTIC POLYSACCHARIDES MONOOXYGENEASE (LPMO) IN COMBINATION WITH PROTEASES

(71) Applicant: SOCIETE DES PRODUITS NESTLE S.A., Vevey (CH)

(72) Inventors: FALICIA GOH, Singapore (SG); ANDREA OLDEN, Singapore (SG); VISHIST KUMAR JAIN, Singapore (SG); CHRISTINA VAFEIADI, Lausanne 26 (CH); MATTHIAS FROMMHAGEN, Lausanne (CH)

(21) Appl. No.: 18/694,891

(22) PCT Filed: Sep. 22, 2022
(86) PCT No.: PCT/EP2022/076394

§ 371 Date: Mar. 22, 2024

(30) Foreign Application Priority Data
Sep. 24, 2021 (EP) 21199001.5

(51) Int. Cl. A23L 7/104; C12N 9/48

(52) U.S. Cl. CPC A23L 7/107; C12N 9/48; C12Y 114/13; C12Y 302/01004

(57) ABSTRACT

The present invention relates a process for preparing a cereal-based extract; said process comprises the steps of: (i) providing a whole grain cereal, (ii) subjecting the whole grain cereal to a single grinding and subsequently combining the whole grain cereal and water, (iii) subjecting the slurry of ground whole grain cereal and water to an enzymatic hydrolysis step, (iv) separating a soluble fraction of the whole grain cereal from an insoluble fraction through filtration, decantation or centrifugation and wherein the enzymatic hydrolysis step makes use of lytic polysaccharides monooxygeneases (LPMOs) in combination with carbohydrate hydrolysing enzymes and at least one protease. The invention also relates to a concentrated cereal-based extract ingredient comprising based on a dry matter basis, 40-90% (w/w) of carbohydrate, 5-20% (w/w) of protein, 0.5-20% (w/w) of total dietary fiber, optionally 1-4% (w/w) of ash and 0-10% (w/w) of fat, and such a extract obtained with the process.

### TECHNICAL FIELD OF THE INVENTION

The present invention relates to a process for preparing a cereal-based extract using improved enzymatic hydrolysis. In particular, the present invention relates to the preparation of a beverage having a beverage ingredient comprising the whole grain cereal-based extract, which whole grain cereal extract has an improved nutritional profile.

### BACKGROUND OF THE INVENTION

Extracts of cereals such as malt extracts have been used as ingredients in beverages, such as in cocoa malted beverages. Brewing processes to produce malt extract are well known in the art. Malt is produced based upon the classic process used by breweries for making beer. In this process cereals such as barley are harvested and dried so that they can be stored until required for use. Barley can either be used directly (un-malted barley) or go through the traditional malting process which involves steeping the grain in water, germinating the grain for a period of days and stabilising the grain by carefully drying it to a low moisture content typically around 5% moisture. During the malting process enzymes for converting the starchy endosperm to soluble components are produced. Enzymes such as α-amylase, β-amylase, β-glucanase, proteases, arabinoxylanases are synthesised in the germinating grain. During malting these enzymes also modify the structure of the cell wall allowing an easier and more complete extraction of the soluble fraction of the cereal during later steps in the process.

The malted/un-malted whole grain cereal is typically ground by milling to a heterogeneous powder which is mixed with water in a ratio of 2-10 (or sometimes even more) parts water to 1 part grain. The initial temperature of the water may be around 50° C. to 60° C. to allow hydrolysis by proteases and cell-wall hydrolysing enzymes. Once this hydrolysis is sufficiently complete the temperature is raised to between 60° C. and 70° C. to allow hydrolysis of the starch by the α- and β-amylases. Once the hydrolysis is complete the temperature of the slurry is increased to around 80° C. to inactivate the enzymes. The slurry is passed through a separating device such as a lauter tun, decanter, plate and frame filter or is centrifuged. The liquid portion is separated from the insoluble/partially soluble material by a process of filtration and leaching. Leaching involves washing the insoluble grain until all of the easily soluble material has been removed. The liquid portion is stabilised by drying to a powder or evaporating to a paste with typical dry-matter content above 78% (w/w).

The powder or paste obtained may be used as an ingredient in the beverage while the, the insoluble fraction of the treated whole grain cereal, such as Barley Spent Grains (BSG) from whole barley which is separated from the mash after filtration, is predominantly sold as animal feed or disposed in land fill.

An alternative approach can be to reduce the generation of insoluble fraction of the treated whole grain cereal, such as BSG, using enzymatic hydrolysis.

Instead of valorising the insoluble fraction of the treated whole grain cereal, such as BSG, for new applications or other usages, a direct optimal yield production from the whole grain cereal would be both cost beneficial as well as environmentally sustainable. As an example, a reduced production of BSG can be obtained by converting insoluble matter in un-malted green barley to soluble matter through enhanced enzymatic hydrolysis of the barley.

### SUMMARY OF THE INVENTION

The present invention reduces the production of insoluble fraction of the treated whole grain cereal, like BSG, by converting insoluble matter in un-malted green barley to soluble matter through enzymatic hydrolysis.

In a first aspect, the invention relates to a process for preparing a whole grain cereal-based extract; said process comprises the steps of:
(i) providing a whole grain cereal,
(ii) subjecting the whole grain cereal to a single grinding and subsequently combining the whole grain cereal and water,
(iii) subjecting the slurry of ground whole grain cereal and water to an enzymatic hydrolysis step
(iv) separating a soluble fraction of the whole grain cereal from an insoluble fraction through filtration, decantation or centrifugation, and wherein the enzymatic hydrolysis step makes use of lytic polysaccharides monooxygenease (LPMO) in combination with carbohydrate hydrolysing enzymes and at least one protease.

In a second aspect, the invention relates to a concentrated whole grain cereal-based extract ingredient comprising, based on a dry matter basis,
40-90% (w/w) of carbohydrate,
5-20% (w/w) of protein,
0.5-20% (w/w) of total dietary fiber,
optionally 1-4% (w/w) of ash and
0-10% (w/w) of fat.

### BRIEF DESCRIPTION OF THE DRAWINGS

FIG. 1 shows experimental results showing yield in the strong wort after enzymatic hydrolysis with the LMPOs in combination carbohydrate hydrolysing enzymes with different proteases.

FIG. 2 shows experimental results showing extraction yield (%) after enzymatic hydrolysis with LMPOs enzyme (Cellic CTEC2) in combination with Alcalase and exopeptidases (Flavourzyme and Protana prime) as compared to other commercially available fiberases.

FIG. 3 shows a schematic representation of the malt extract production process.

The present invention will now be described in more detail in the following.

### DETAILED DESCRIPTION OF THE INVENTION

It has surprisingly been found that by treating a whole grain cereal in a special manner it becomes possible to improve the nutritional property of cereal extract along with yield improvement compared to Reference.

According to the invention the process comprises the steps of:
(i) providing a whole grain cereal,
(ii) subjecting the whole grain cereal to a single grinding and subsequently combining the whole grain cereal and water,
(iii) subjecting the slurry of ground whole grain cereal and water to an enzymatic hydrolysis step to break down macromolecular elements being proteins, polysaccharides and lipids in the whole grain to smaller units,
(iv) separating a soluble fraction of the whole grain cereal from an insoluble fraction through filtration, and wherein the enzymatic hydrolysis step makes use of lytic polysaccharides monooxygenease (LPMO) in combination with at least one protease.

For example, the process comprises the steps of
(i) providing a whole grain cereal,
(ii) subjecting the whole grain cereal to a single grinding and subsequently combining the whole grain cereal and water,
(iii) subjecting the slurry of ground whole grain cereal and water to an enzymatic hydrolysis step,
(iv) separating a soluble fraction of the whole grain cereal from an insoluble fraction through filtration, decantation or centrifugation, and
wherein the enzymatic hydrolysis step makes use of lytic polysaccharides monooxygenease (LPMO) in combination with carbohydrate hydrolysing enzymes and at least one protease.

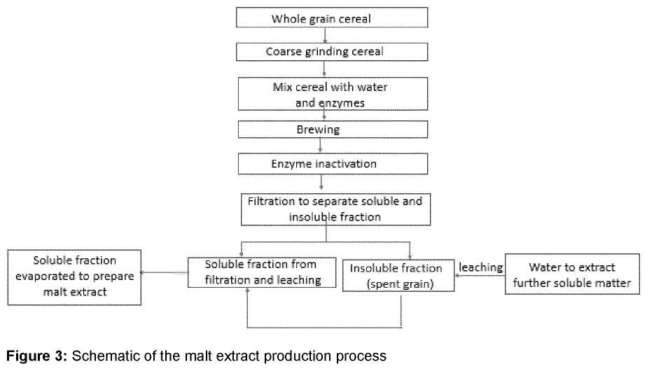

The process according to the present invention may be as outlined in the flowchart in FIG. 3.

In the present context the term “grinding” means any physical destruction of the whole grain with the purpose to have the macromolecular structure of the whole grain more available, for example to water and/or enzymes.

The grounded whole grain cereal may then be mixed with water and enzymes to modify the whole grain cereal. When the whole grain cereal has been sufficiently modified the enzymes are inactivated and the insoluble fraction of the whole grain cereal may be separated from the soluble fraction of the whole grain cereal. The soluble fraction is then evaporated to high total solids (60%-90%) to provide a beverage ingredient or is mixed directly into the beverage.

In the present invention the term “whole grain cereal-based extract” relates to an extract obtained from treating cereals by the process according to the present invention. Preferably, the whole grain cereal-based extract comprises at least part of the insoluble fraction of the whole grain cereal.

In the present context carbohydrates, protein, and dietary fiber comprises also fragment hereof.

In the present context the term “insoluble fraction” relates to a fraction obtained from the whole grain cereal comprising the insoluble fibres, i.e. the insoluble dietary fibres that are not fermented in the large intestine or only slowly digested by the intestinal microflora. Examples of insoluble fibres include celluloses, hemicelluloses, resistant starch type 1 and lignins. Health authorities recommend a consumption of between 20 and 35 g per day of fibres, depending on the weight, gender, age and energy intake.

The present invention relates to the use of an enzymatic hydrolysis step makes use of lytic polysaccharides monooxygenease (LPMO) in combination with carbohydrate hydrolysing enzymes and at least one protease

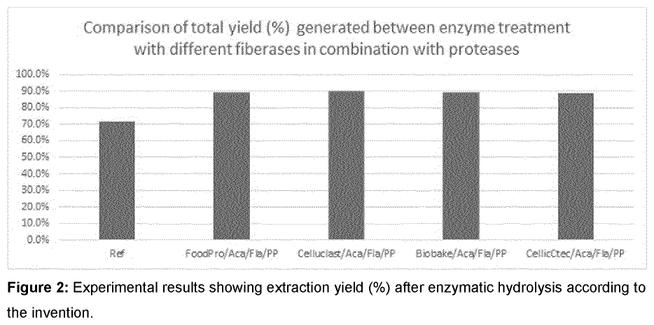

Several advantages of using LPMO enzymes in accordance with the invention to obtain the soluble fraction with improved yield and nutritional profile exist:
I. The amount of soluble fraction obtained with improved enzymatic treatment is higher compared to Reference process. Based on FIG. 2, around 90 kg of soluble dry matter can be obtained per 100 kg of dry cereal matter compared to only 71 kg of soluble dry matter per 100 kg of dry cereal matter. This shows that more of the insoluble fraction present in the whole grain cereal can be solubilized through improved enzymatic process and appear in the product.
II. An increase in the protein content and a reduction in total carbohydrate content may be provided in the final product.

In this invention, a combination of enzymes comprising of fiberases and proteases is used for increasing the extraction yield of whole grain cereals, like un-malted barley. of the combination of fiberase enzymes such as xylanases, cellulases and glucanases with commercial proteases (endo and/or exo proteases) solubilize recalcitrant fibers and proteins present in the plant cell wall of whole grain cereals. Cellulose is more resistant to hydrolytic cleavage compared to other fibers. such as xylans, due to its crystallinity. The conventional cellulases present in the enzymes used for the hydrolysis of whole grain cereals, like green barley, are currently inefficient. As alternative, lytic polysaccharide monooxygenase (LPMO) in combination with cellulases were used to improve the degradation cellulose, and thereby the extraction yield of whole grain cereals.

In the present invention it has been found that by using LMPOs alone with other cellulases does not lead to a significant improvement of the extraction yield of whole grain cereals, like the current un-malted barley. In the present invention it has been found that for significant increase in extraction yield it is however imperative to add hydrolytic enzymes in combination with proteases (endo and exo peptidases) and LPMOs (e.g. provided as part of Cellic CTEC 2). It has been shown that LPMOs (e.g. provided in the form of Cellic CTEC 2) led to marginal increases in extraction yield compared to the Reference yield whereas inclusion of complete set of proteases leads to a yield improvement of around ˜10%.

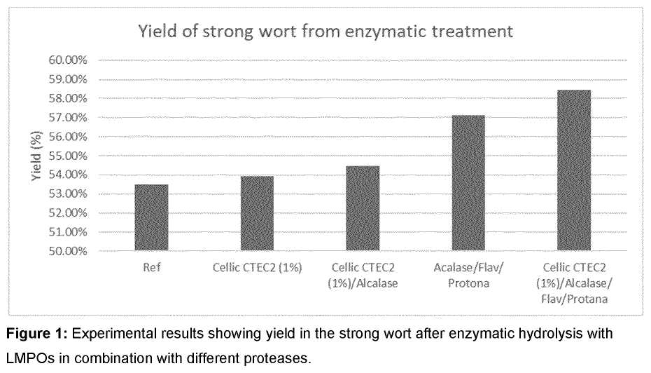

FIG. 1 shows a comparison to Reference based on collection of 235 g of strong wort. In the present context the yield is determined by calculating the total dry matter in 235 g of hydrolysate and dividing this with the total initial dry matter. This is achievable through synergistic action of LPMO with hydrolytic enzymes and complete set of proteases which can in turn impact the nutritional profile of the final cocoa and malt beverage.

Table 1 below compares the composition of Reference and improved version of the malt extract. The improved malt extract had an 8.2% reduction in total carbohydrates and 2 times increase in protein content as compared to current malt extract. Although it has been found that the addition of the fiberase do not significantly affect composition of high molecular weight dietary fiber, it allows accessibility of the proteases for their activities. Hence, not only has it been found that an increase in yield lead to decrease in spent grain production but also improved the overall nutritional values of the malt extract. The percentage of yield with the combination is also higher or equal to that when other commercial fiberases such as Food pro CBL and Celluclast 1.5 L, in combination with the proteases were use, see FIG. 2.

TABLE 1
Nutritional composition of current versus improved malt
extracts based on dry matter. Improved malt extract had
8.2% reduction in total carbohydrates and protein content
was doubled as compared to current malt extract.

| Nutritional composition | Current | Improved |
|---|---|---|
| (g/100 g in malt extract)) | Malt Extract | malt extract |
| Fat | 0.1 | 0.1 |
| Total Carbohydrates | 73.3 | 67.3 |
| Total Dietary Fiber | 8.96 | 1.13 |
| (HMWDF + LMWSDF) | | |
| High Molecular Weight | 1.36 | 1.13 |
| Dietary Fiber (HMWDF) | | |
| Low Molecular Weight Soluble | 7.6 | <0.5 |
| Dietary Fiber (LMWSDF) | | |
| Protein | 4.14 | 8.33 |
| Ash | 1.03 | 1.56 |
| Moisture | 21.4 | 22.7 |
| Total | 99.97 | 99.99 |

FIG. 2 shows the experimental results showing extraction yield (%) after enzymatic hydrolysis according to the invention with LMPOs enzyme (Cellic CTEC2) in combination with Alcalase and exopeptidases (Flavourzyme and Protana prime) as compared to other commercially available fiberases.

In an embodiment of the present invention the soluble fraction may be a malt extract.

The starting material in the present process may be a whole grain cereal. Whole grain cereal is a product made from cereal grains comprising the entire edible parts of a grain, i.e. germ, endosperm and bran.

In the present context the term “cereal” relates to monocotyledonous plants of the Poaceae family (grass family) cultivated for their edible, starchy grains.

In an embodiment of the present invention the whole grain cereal is selected from the group consisting of barley, oat, brown rice, wild rice, bulgur, corn, millet, sorghum, spelt, triticale, rye, wheat, wheat berries, teff, canary grass, Job's tears, fonio and pseudocereals. Plant species that do not belong to the grass family, but also produce starchy seeds or fruits that may be used in the same way as cereal grains, are called pseudocereals. Examples of pseudocereals include amaranth, buckwheat, tartar buckwheat and quinoa.

In another embodiment of the present invention the terms “cereal” and/or “whole grain cereal”, include both cereal and pseudocereals. Preferably, the terms “cereal” and/or “whole grain cereal” do not include pseudocereals.

In a preferred embodiment of the present invention the whole grain cereal is an un-germinated/un-malted whole grain cereal. In one embodiment, the un-malted whole grain cereal is derived from barley or oat.

Preferably, the process according to the invention comprises a step where the soluble fraction obtained from the separation in step (iv) is concentrated. It is preferred that the concentrate comprises at least 30% and up to 97.5% (w/w) of the soluble fraction after concentration.

In one embodiment of the process of the invention the slurry of wholegrain cereal in step (ii) is heated to gelatinise the starch. Preferably, the slurry of whole grain cereal is heated to a temperature in the range of 50-70° C. This has the advantage of making the substrates more available for enzymatic hydrolysis.

One advantage of the insoluble fraction provided by the process described in the present invention may be that the suspension properties of the insoluble fraction are improved. This improvement is considered relative to the suspension properties of an unprocessed insoluble fraction, e.g. having a main particle size above 100 μm.

Following the grinding process the ground whole grain cereal is subjected to a hydrolysis of the macromolecular elements of the whole grain cereal. For example, the ground whole grain cereal may be subjected to a hydrolysis of carbohydrates and/or protein, and/or lipid, and/or other organic components (for example, polyphenols).

In an embodiment of the present invention the hydrolysis, for example, of carbohydrates and/or protein (in step (iii)), is an enzymatic modification. In an embodiment, the modification is by enzyme degradation. Preferably, the enzymatic modification may be performed at a temperature in the range of 10° C.-122° C., preferably in the range of 20-100° C., such as in the range of 20-40° C. or in the range of 40° C.-65° C.

In another embodiment of the present invention the hydrolysis, for example, of carbohydrates and/or protein (in step (iii)), may be performed until substantially complete modification of the starch has taken place. The term “substantially complete modification” relates to at most 10% of the original starch content, may be remaining after modification, such as at most 5%, preferably at most 2%, more preferably at most 1%, such as at most 0.5% of the original starch content, may be remaining after modification.

The hydrolysis, for example, of carbohydrates and/or protein in step (iii), may be performed by one or more endogenous enzyme(s) and/or by the addition of one or more exogenous enzyme(s) or by the combination thereof.

Preferably in the process according to the invention the hydrolysis is carried out with additional enzymes with different functionalities such as alpha amylase, other fiberases and lipases.

In respect of the one or more exogenous enzyme(s), such exogenous enzyme(s) may be selected from the group consisting of proteases (endo and exo proteases), dextrinases, cell-wall hydrolyzing enzymes such as—LPMOs, amylases and amyloglucosidases, fragments thereof and any combination thereof. Preferably, a mixture of several of the above enzymes may be used.

Advantageously, the fiberase contain at least cellulases. Preferably, the cellulases possess additional activities selected from the group consisting of endo-glucanases, cellobiohydrolases and beta-glucosidases.

In another embodiment of the present invention at least one of the endogenous enzyme(s) and/or the exogenous enzyme(s) is a protease and/or an amylase. The protease may be active in alkaline, neutral and/or acid pH conditions.

In yet an embodiment of the present invention the proteases contain at least one endoprotease and two exopeptidases.

It is preferred that the exopeptidases are selected from the group consisting of carboxypeptidases, aminopeptidases or combination thereof.

The amylase may preferably be an alpha-amylase, such as 1,4-α-D-glucan glucanohydrolase or glycogenase, a beta-amylase, such as 1,4-α-D-glucan maltohydrolase or saccharogen amylase, a gluco-amylase, such as amyloglucosidase or Exo-1,4-α-glucosidase or any combination hereof.

When the hydrolysis for example, of carbohydrates and/or protein, has reached a substantially complete modification the process may further comprise a step of inactivating the enzymatic activity. This inactivation may be performed by changing the temperature to a temperature in the range of 40-130° C., preferably in the range of 75-85° C. Preferably the inactivation may be performed for a period of time of at least 15 seconds, such as at least 30 seconds, e.g. at least 1 minute, such as at least 5 minutes, e.g. at the least 10 minutes, such as at least 20 minutes, e.g. at least 30 minutes.

Hydrolysis of the different macromolecular elements of the whole grain cereal can also be achieved by any other means known in the art, such as chemical modification, e.g. acid or heat-induced hydrolysis.

When the hydrolysis has ended, and the soluble parts of the whole grain cereal have been liberated from the insoluble parts the soluble fraction and insoluble fraction may be separated. Thus, in an embodiment of the present invention the separation of the soluble fraction from the insoluble fraction (in step (iv)) may be selected from the group consisting of filtration, centrifugation, decanting and a combination thereof.

Depending on the application of the soluble fraction it may be further treated to provide different fractions comprising specific components or it may be concentrated. Preferably the soluble fraction obtained from the separation (in step (iv)) is concentrated. The concentrate may comprise at least 10% and up to 97.5% (w/w) of the soluble fraction before concentration. The final concentrate may be in the form of a liquid, a gel or a powder.

Optionally a filtration may be used in the process according to the invention. The process aids may be diatomite, perlite, celite, cellulose or grain hulls.

In yet another embodiment of the LPMO enzyme treated whole grain cereal based fraction comprises 8% (w/w) protein and 67% (w/w) carbohydrate.

In another embodiment of the present invention the beverage ingredient comprises at most 70% (w/w) protein, such as at the most 50% (w/w) protein, e.g. at the most 20% (w/w) protein, such as at the most 2% (w/w) protein, e.g. at the most 1% (w/w) protein.

In another embodiment of the present invention the beverage ingredient comprises at least 5% (w/w) protein, such as at least 10% (w/w) protein, e.g. at least 25% (w/w) protein, such as at least 50% (w/w) protein, e.g. at least 60% (w/w) protein.

It may be advantageous that the present beverage ingredient may be a high sugar beverage ingredient or a low sugar beverage ingredient. When the beverage is a high sugar beverage ingredient the beverage ingredient comprises above 50% (w/w) sucrose and at most 95% (w/w) sucrose, such as at most 85% (w/w) sucrose, e.g. at most 75% (w/w) sucrose, such as at most 65% (w/w) sucrose. When the beverage ingredient is a low sugar beverage ingredient the beverage ingredient comprises at most 50% (w/w) sucrose, such as at most 40% sucrose, e.g. at most 25% sucrose, such as at most 15% sucrose, e.g. at most 10% sucrose, such as at most 5% sucrose, e.g. 0% sucrose.

To control and/or improve the sensory impression of the beverage and the beverage ingredient the beverage ingredient comprises a flavour component. In an embodiment of the present invention the flavour component may be selected from the group consisting of cocoa, coffee, fruit, malt, soya, tea, vegetable, and any combination thereof.

The beverage ingredient may also comprise a fat component. In an embodiment of the present invention the fat component may be a vegetable fat component, a fish oil component or a combination thereof.

The beverage ingredient may further comprise a milk component, such as a skimmed milk component and/or milk component.

Depending on the application of the beverage ingredient and the production conditions, the beverage ingredient may be in the form of a liquid, a concentrate, a puree or a powder.

An embodiment of the invention relates to a concentrate cereal-based extract ingredient comprising based on a dry matter basis, 60-90% (w/w) of carbohydrate, 5-15% (w/w) of protein, 0.5-5% (w/w) of total dietary fiber fraction, optionally 1-4% (w/w) of ash and 0-1% (w/w) of fat fraction.

An embodiment of the invention relates to a concentrate cereal-based extract ingredient comprising based on a dry matter basis, 40-90% (w/w) of carbohydrate, 5-20% (w/w) of protein, 0.5-20% (w/w) of total dietary fiber fraction, optionally 1-4% (w/w) of ash and 0-10% (w/w) of fat fraction.

An embodiment of the invention relates to a concentrate cereal-based extract ingredient comprising based on a dry matter basis, 55-70% (w/w) of carbohydrate, 5-15% (w/w) of protein, 0.5-5% (w/w) of total dietary fiber fraction, optionally 1-4% (w/w) of ash and 0-1% (w/w) of fat.

Preferably, the concentrate cereal-based extract ingredient according to the invention wherein the ingredient comprises, on a dry matter basis, 25-40% (w/w) of concentrated cereal-based extract, 20-40% (w/w) of skimmed milk powder, 10-20% (w/w) of sucrose, 5-20% (w/w) of cocoa powder and 0-5% (w/w) of fat.

In a preferred embodiment of the present invention the beverage ingredient comprises 25-45% (w/w) of a mixture of malt extract, preferably 34-38% (w/w), 15-25% (w/w) of a skimmed milk powder, preferably 18-22% (w/w), 10-20% (w/w) carbohydrate, preferably 14-18% (w/w), 10-20% (w/w) cocoa, preferably 12-15% (w/w), and 5-15% (w/w) of a fat component, preferably 8-12% (w/w). The carbohydrates are preferably sucrose.

In the present case, malt extract may be considered as soluble fraction of the modified whole grain cereal.

The beverage ingredient may preferably be used for the preparation of a beverage. In particular, the beverage ingredient may be used for the preparation of a beverage having and/or an improved nutritional value.

In an additional aspect, the invention relates to a process for preparing a beverage having improved organoleptic and/or improved nutritional value, said process comprises the steps of:
(a) providing concentrate cereal-based extract ingredient to claims 10-14,
(b) mixing the ingredient of step (a) with a liquid component and obtaining the beverage. Liquid component may be selected from the group consisting of water, milk, fruit juice, vegetable juice or any combination thereof.

In a further aspect, the invention relates to a beverage consisting of:
(1) a liquid component and an ingredient component as discussed above. In a preferred embodiment the beverage provides at least 14% of the total energy of the beverage is provided by protein, less than 21% of the total energy of the beverage is provide by fat, at least 62% the total energy of the beverage is provided by carbohydrates, and at least 2% of the total energy of the beverage is provided by total dietary fiber.

The milk component may be selected from the group consisting of whole milk, whey fractions, casein, any combination hereof.

Hence, a beverage may be provided consisting of:
(1) a liquid component; and
(2) a beverage ingredient as described in the present invention.

In the present context the term “beverage” refers to a composition in the form of a dry powder, a slurry or a liquid. It is to be understood that the dry powder may be reconstituted in any applicable liquid suitable for consumption. The slurry or the liquid may be further diluted using any applicable liquid suitable for consumption.

In the present context the term “(w/w)” relates to a weight by weight ratio of a compound or product on a dry-matter basis unless any this else is stated.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1-Enzymatic Hydrolysis of Cereal Using Enzymes from Biofuel Industry to Improve Nutritional Properties of Extract

Cereal grist obtained after a single milling step is mixed with water and subjected to enzymatic hydrolysis step. Enzymes, mostly cellulases from biofuel industry, help to hydrolyze crystalline cellulose which is usually difficult to break down using conventional cellulases. Furthermore, these cellulase work in synergy with proteases to improve the yield of the process. After hydrolysis the slurry goes through a filtration step to obtain extract which undergoes evaporation step to increase total solids content. This malt extract can have higher protein content and lesser carbohydrate content.

### Example 2-Cacao Beverage with Improved Protein in Malt Extract

A powder that is dosed at 15 g per 100 ml water and with 1 serving being defined as 28-30 gram. A powder containing 30-40% modified whole grain and bran malt extract preferably about 38% 15-40% skimmed milk powder preferably about-21%), 10-20% sugar (ideally 15%), 10-20% cocoa (preferably 12%) and 5-15% fat (10%), of which the whole grain and bran malt extract contains around 1-5% valorised spent grain which consists of ca. 50% insoluble fibers, ca. 25% protein and ca. 25% carbohydrates. The beverage has a caloric value below 255 kcal per serving and is intended to become consumed as a major part of the meal (e.g. breakfast).

1. A process for preparing a cereal-based extract; said process comprises the steps of:
(i) providing a whole grain cereal,
(ii) subjecting the whole grain cereal to a single grinding and subsequently combining the whole grain cereal and water,
(iii) subjecting the slurry of ground whole grain cereal and water to an enzymatic hydrolysis step,
(iv) separating a soluble fraction of the whole grain cereal from an insoluble fraction through filtration, decantation, or centrifugation, and
wherein the enzymatic hydrolysis step makes use of lytic polysaccharides monooxygeneases (LPMOs) in combination with at least one protease.

2. A process according to claim 1, wherein the whole grain cereal is un-malted whole grain cereal, derived from barley or oat.

3. A process according to claim 1, wherein the soluble fraction obtained from the separation in step (iv) is concentrated.

4. A process according to claim 3, wherein the concentrate comprises at least of 30% and up to 97.5% (w/w) of the soluble fraction after concentration.

5. A process according to claim 1, wherein the slurry of wholegrain cereal in step (ii) is heated to gelatinise the starch.

6. A process according to claim 1, wherein the hydrolysis is carried out with additional enzymes with different functionalities.

7. A process according to claim 6, wherein the enzymes comprise proteases contain at least one endoprotease and two exopeptidases.

8. A process according to claim 7, wherein exopeptidases are selected from the group consisting of carboxypeptidases, aminopeptidases and combination thereof.

9. A process according to claim 6, wherein fiberase contain at least cellulases.

10. A process according to claim 9, wherein cellulases possess additional activities selected from the group consisting of endoglucanases, cellobiohydrolases and beta glucosidases.

11. A concentrate cereal-based extract ingredient comprising based on a dry matter basis,
40-90% (w/w) of carbohydrate,
5-20% (w/w) of protein,
0.5-20% (w/w) of total dietary fiber,
1-4% (w/w) of ash and
0-10% (w/w) of fat fraction.

12. (canceled)

13. A concentrate cereal-based extract ingredient according to claim 11, wherein the ingredient comprises, on a dry matter basis, 25-40% (w/w) of concentrated cereal-based extract, 20-40% (w/w) of skimmed milk powder, 10-20% (w/w) of sucrose, 5-20% (w/w) of cocoa powder and 0-5% (w/w) of fat.

14-17. (canceled)
